(19) United States
(12) Patent Application Publication
(10) Pub. No.: US 2016/0038088 A1
(43) Pub. Date: Feb. 11, 2016

(54) SYSTEMS AND DEVICES FOR MEASURING, CAPTURING, AND MODIFYING PARTIAL AND FULL BODY KINEMATICS

(71) Applicants: David Lari, San Francisco, CA (US); Luke Clauson, Redwood City, CA (US); Mike Schaller, Redwood City, CA (US)

(72) Inventors: David Lari, San Francisco, CA (US); Luke Clauson, Redwood City, CA (US); Mike Schaller, Redwood City, CA (US)

(21) Appl. No.: 14/815,851

(22) Filed: Jul. 31, 2015

Related U.S. Application Data
(60) Provisional application No. 62/031,809, filed on Jul. 31, 2014.

(51) Int. Cl. A61B 5/00; A61B 5/11

(52) U.S. Cl. CPC A61B 5/6898; A61B 5/11; A61B 5/7278; A61B 5/742; A61B 5/0024; A61B 5/7246; A61B 5/0022; A61B 2562/0219; A61B 2562/0247; A61B 2562/0261; A61B 2560/0475; A61B 2560/0487

(57) ABSTRACT

Devices, systems, and methods are disclosed for measuring, capturing, and modifying the motion of a body, for providing visual comparisons, metrics of comparison, and for generating motion standards. Motion standards may be manually or algorithmically modified to a different desired motion standard. The motion standards may be generated during motion capture and may be used as references for comparing amongst other motion standards or captured motions. Comparisons may generally be used for training and improving upon motions, such as athletic related motions, rehabilitation related motions, and the like. In certain embodiments data capture is done primarily by sensor units worn on a body which communicate amongst each other and with a secondary processing unit, which analyzes the data, provides means of comparing and modifying a captured motion, and which stores or relays such data to a tertiary device, such as a remote database.

### CROSS-REFERENCES TO RELATED APPLICATIONS

This application claims priority to U.S. provisional patent application No. 62/031,809, filed on Jul. 31, 2014, the full disclosure of which is incorporated herein by reference.

### FIELD OF THE INVENTION

This disclosure relates generally to wearable sensing devices and particularly to a system and method for measuring, capturing, and modifying a motion profile using wearable devices.

### DESCRIPTION OF THE RELATED ART

Wearable technology (“wearables”) has seen dramatic growth in recent years—for instance, some estimates show wearable smart bands (products from FitBit, Jawbone, Nike, Swift) alone heading towards 350% growth in 2014, with shipment projections of 8 million units for the year. Wearables have proven their worth in providing users with a general indicator of activity, and in providing data and a means for historical comparison with some level of metric tracking. Another class of wearable sensor such as the devices from Notch, Xens, Radio6ense, Invensense, APDM, Biosens, Apple, Captapult Sports etc. track wearer's (including team members') motion and are being used for motion- and other user metric-tracking for movie making, video gaming, virtual reality, sports motion recording, team coordination, physical therapy, surgical recovery etc. These are a natural extension of the wired and more bulky wireless sensors used in university kinematic motion labs for many decades. This class of wireless wearable sensor is often relatively compact, wireless, communicates from sensor to sensor and to a host and often has real time data processing on board. All the products of the above listed companies have the ability to track and/or describe motion in some capacity. Some allow for motion data capture (APDM, etc.), though none have capability of modifying captured motion, nor do any have ability to dynamically compare a modified motion profile to a recorded motion in real-time.

Despite the plethora of wearable, data producing motion capture sensors and systems, there is no solution that analyzes and compares repetitive motions with previously defined motion standards automatically and allows social network sharing and comparison of such standards and comparisons.

Additionally, none of the systems have the ability to perform motion profile modification. Thus there is a need for improvement in existing systems and products.

### SUMMARY OF THE INVENTION

Systems and devices for measuring, capturing, and modifying an individual's partial or full body kinematics, by at least one wearable sensor, are disclosed. The system comprises at least one sensor configured to transduce and provide kinematic information about a movement of the user's body. The system further comprises a computer interface device with processor memory and a display configured to analyze the kinematic information about said movement. The computer interface device is configured to store a pre-determined motion standard, provide a means of modifying a captured motion, and compare the kinematic information to said motion standard, and output the results of the comparison through the display.

In embodiments of the system the sensor is a gyroscopic sensor, an accelerometer, a strain sensor, a resistive, capaciative or a pressure sensor. In one embodiment of the system the motion standard is determined by recording the user's own movement. In one embodiment of the system the motion standard is determined by manual or automatic programmatic manipulation of a recorded movement. In one embodiment of the system the motion standard comprises a database of other users' movement. In one embodiment the motion standard is peer-to-peer shared between users. In some embodiments of the system the analyzing kinematic information compensates for body differences between that of the motion standard and that of the user.

In various embodiments of the system the computer interface device comprises a mobile phone, a laptop computer, or a wearable computer. In various embodiments of the system the sensors communicate to the computer interface device via wired or wireless communication. In some embodiments the wireless communication uses a wireless communication protocol selected from one of radio frequency, Bluetooth, Zigbee, Ultra Wide Band or WiFi.

A method of measuring, capturing, and modifying a user's body kinematics is disclosed comprising receiving inputs from sensors relating to movement of a user, analyzing the sensor inputs to determine kinematic state information of the user, comparing the kinematic state information to that of a motion standard, and representing the result of the comparison to the user via an interface. In one embodiment of the method the motion standard comprises a database of the user's own movement. In one embodiment of the method the motion standard comprises a database of movement of individuals other than the user.

### BRIEF DESCRIPTION OF THE DRAWINGS

The invention has other advantages and features that will be more readily apparent from the following detailed description of the invention and the appended claims, when taken in conjunction with the accompanying drawings, in which:

FIG. 1A shows one embodiment of a system for measuring and tracking a person's body kinematics.

FIG. 1B shows embodiments of the system in which sensors are used in a shoe for measuring and tracking a person's body kinematics.

FIG. 1C and FIG. 1D show embodiments of the system in which a smartphone (FIG. 1C) and a laptop computer are used to display analysis of a person's body kinematics. FIG. 2 shows a method of measuring and tracking a person's body kinematics according to one embodiment.

FIGS. 3A and 3B illustrates exemplary embodiments in which analyzed foot movement of a person is compared against a standard to derive useful information.

FIGS. 4A, 4B, and 4C illustrate exemplary embodiments in which analyzed golf swing motion is captured as a motion standard and modified to a new motion standard, both of which are shown alongside live motion.

### DETAILED DESCRIPTION

Definitions

Kinematic information: Of or relating to any metrics, descriptors, or the like which describe a motion of a physical body, in part or in whole, qualitatively or quantitatively.

Partial body kinematics: Of or relating to any motion which generally describes all primary motion of a subset of a physical body, i.e. motion of a subset of a body's comprising elements, where comprising elements are generally defined as section of a body next to or between joints, linkages, or equivalent.

Full body kinematics: Of or relating to any motion which generally describes all primary motion of an entire physical body, i.e. motion of most of a body's comprising elements, where comprising elements are generally defined as section of a body next to or between joints, linkages, or equivalent.

Motion standard: A path (or collection of paths) of a point (or collection of points) through space and time, as defined by a series of space-time coordinates, or equivalent, which generally describe the partial or complete motion of a body and/or of a body's comprising elements, and which generally, but not necessarily, are linked with specific motion activities.

A motion standard may be derived from an unaltered motion of a captured motion, a completely artificial motion defined empirically/algorithmically/manually, or the result a combination of the two, such as manipulation/modification of a captured motion. Modification/manipulation of a motion may come in the form of adjusting various coordinates and/or time points in a motion standard, or a drag and drop system of a wireframe or equivalent model of a motion standard at various parts of the motion standard, or equivalent, all of which may have the ability of software to algorithmically smooth the data. A stored motion standard may be displayed alongside a video capture of a similar motion being performed, with software algorithmically determining when and where to overlay the captured data (i.e. side by side or a direct overlay). Captured data which is similar to or used for creating a motion standard may be used to feed into such a motion standard, for further dynamic modification or manipulation, artificially by the software or manually.

Motion standards which may be individually defined, or downloaded from a database are disclosed. When performing physical activities, technique plays a large role in an individual's performance and health, which can which can be adjusted properly with the right information by repetitive motion training. This repetitive motion training is often required to perfect one's athletic form. For example, the Notch device can be used to record a motion then provide haptic feedback through the sensor to the user based on programmatic decisions surrounding the motion. Their solution however, does nothing to compare a user's motion data to an external standard. No predicate system allows dynamic motion feedback against a professional athlete's motion or against a motion recorded under, and possibly modified under the eye of a trainer or coach. Systems such as the Notch could ostensibly be used as motion training tools, like many other similar devices, but no devices support, currently allow or purport to allow dynamic comparison and feedback of motion compared to an external modified motion standard. Such a baseline can either be a trained motion, or a programmed motion. Such as device may additionally monitor electro-physiological signals, as well as body heat, perspiration, etc. which could be programmatically used to further assess performance or conditions. When multiple sensors/gauges are used in concert, they may communicate with one another, additional sub-sensors such as in-shoe sole strain gages etc. or data capture devices or arrays for more systematic measuring and tracking.

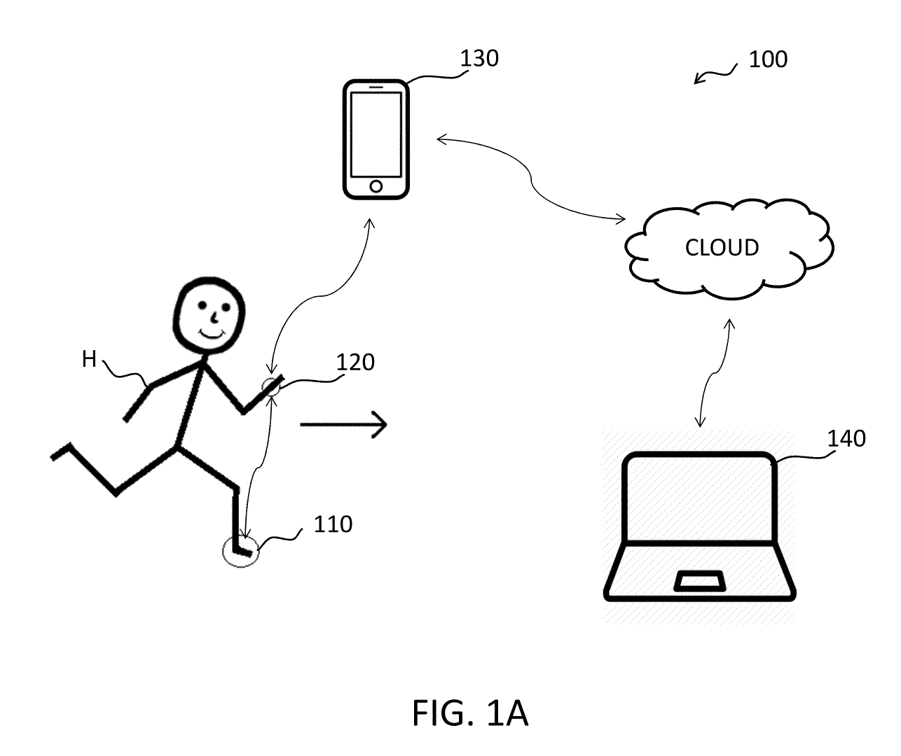

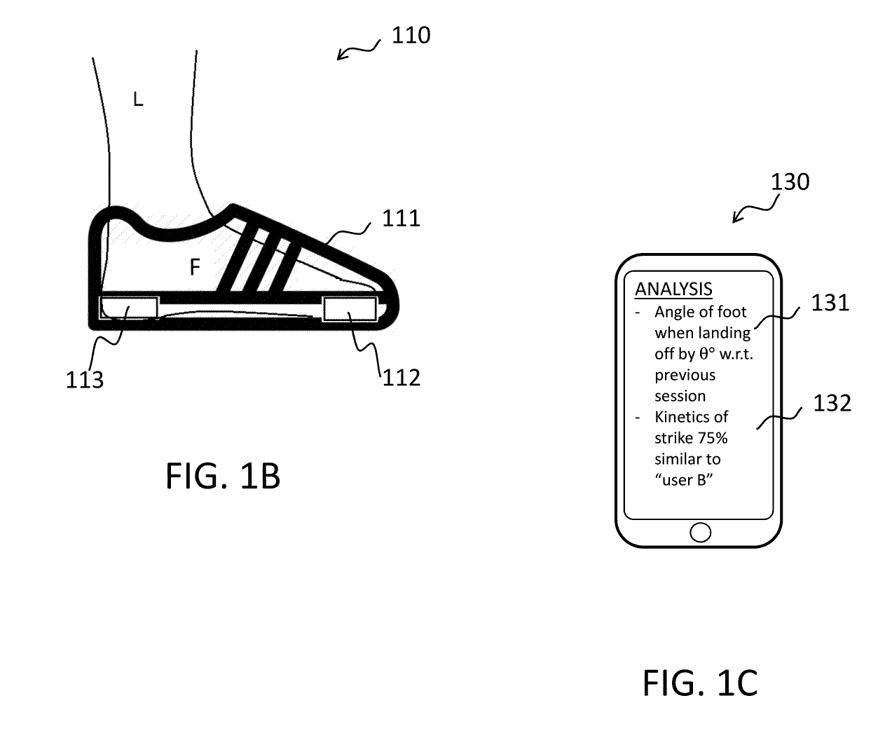

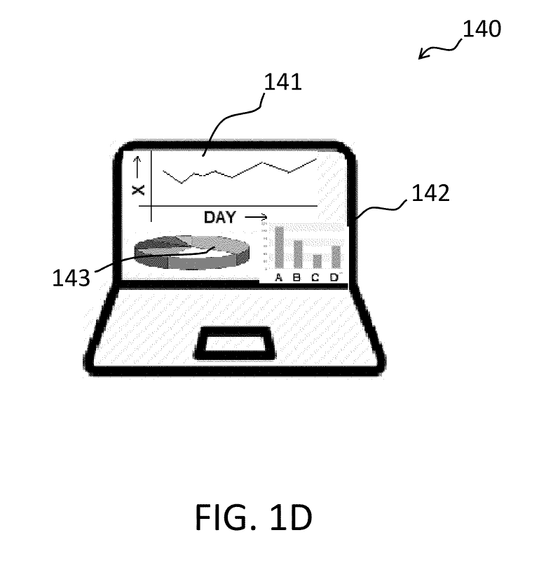

The invention discloses devices, systems and methods for measuring, capturing, and modifying partial and full body kinematics of a user according to the embodiments set forth herein. In one embodiment, a system for achieving the aforesaid objectives is disclosed in FIG. 1A. As shown in FIG. 1A, the system 100 comprises wearable sensor unit 110 worn by a user H, that communicates with a secondary computing device 130 either directly or via a primary communication device 120. Wearable sensor unit 110 is configured to transmit data to communication devices 120 and 130, either through wired or wireless means. Data from sensor unit 110 captured by computing device 130, is configured to be dynamically modified or manipulated, artificially by the software or manually by the user. Optionally, the computing device 130 may communicate with the Cloud for accessing data or to an additional computing and display device such as a laptop 140.

In one embodiment the wearable sensor unit 110 is shown in further detail in FIG. 1B, where user wears a sensor or host of sensors held within a garment such as a shoe 111 on the users foot F. The sensors may comprise a ventrally located sensor 112 and a dorsally located sensor 113, which collect and transmit motion related data to a secondary computing device 120 and then to a secondary graphical computing device 130, alternately the sensors may transmit directly to the secondary graphical computing device. All collected data may be sent to a remote database, such as a cloud server, which stores the data and may distribute the data back to the user's secondary device 120, 130 or to a remote computing device 140 accessed by other users. Any of the shown computing devices may be capable of translating the captured data into a motion standard file, and modifying such motion standards. Metrics of analysis on the computing devices may come in the form of numerical comparison among the users own motion standards 131 or the motion standards of other users 132, or in the form of graphs 141 or charts 142, 143.

In the embodiment of the device is shown in FIG. 1B, the sensors 112 and 113 comprise housing that is small in form, water- and moisture-proof, has features for modular connections to other hardware, and comprises all of the on board electronics, such as, but not limited to:

Sensor(s)/gauge(s)

Microprocessor(s)

Memory unit(s)

Communication antennae(s)

Power source(s)

In one embodiment a method of for measuring, capturing, and modifying partial and full body kinematics of a user is disclosed according to FIG. 2. As shown in the figure, the flow of events begins with sensors collecting and sending motion data, which are received by a secondary computing device 130 in step 201. The secondary computing device 130 determines the type of motion based on the analysis of the information received in step 202. The motion is then compared to a motion standard of a similar motion in step 203, and the results are presented to the user through an interface in step 204.

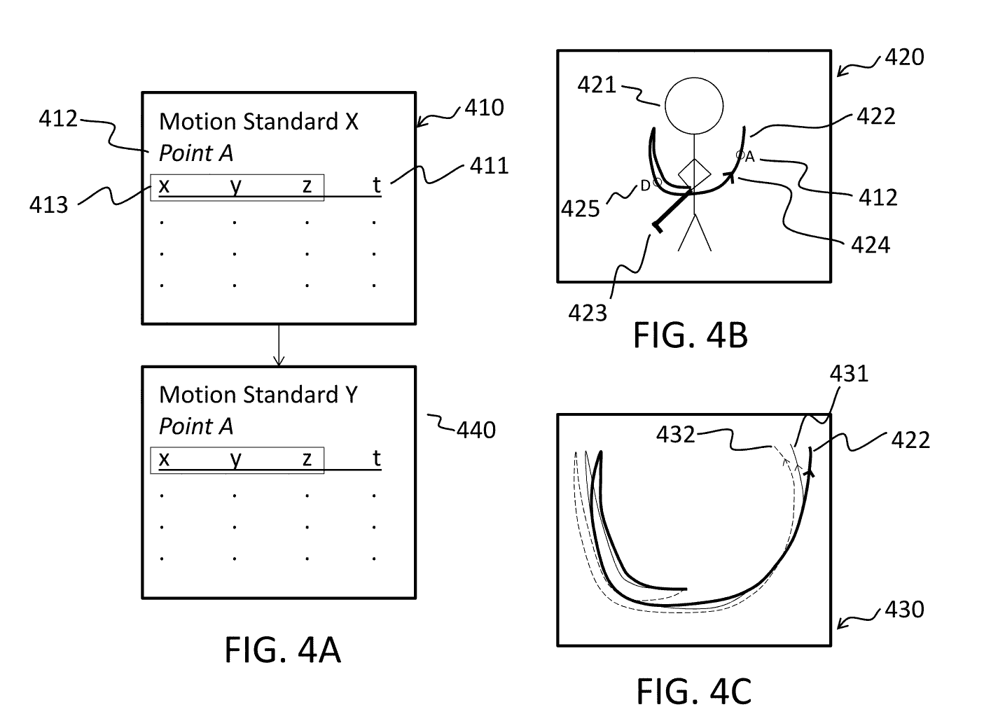

In another embodiment, as disclosed in FIG. 4 an original motion is captured and modified, and live motion is then captured and overlaid on the original and modified motions. As shown in the figure, a visual representation of a golfer 421 swinging a golf club 423 is shown with the motion profile X 422 of the golfer's hands, an arrow 424 showing the direction of motion, and by which motion is tracked and captured by a sensor module or the like. The shown motion profile is converted to a motion standard, shown as Motion Standard X 410, which comprises all space coordinates 413 with corresponding time coordinates 411 for all tracked points of motion capture, as shown a single point's motion is tracked, Point A 412. Modification to motion standard X may be done in any manner described herein, in one manner in particular specific points, such as Point D 425 along the motion profile may be dragged-and-dropped, whereby the motion profile 422 is treated as a spline or equivalent and the modified motion profile is smoothed accordingly, typically algorithmically. The modified motion profile is saved as a new Motion Standard Y 440. A visual representation of motion profile Y 432 of Motion Standard Y 440 is shown overlaid with the motion profile X 422 of Motion Standard X 410, which is shown with a live (i.e. real-time) or newly recorded motion profile Z 431, on a secondary computing device with a screen, or equivalent. The overlaid motion profiles 430 are shown statically, though in actuality, they may be played as a video, using the associated time coordinates 411.

The onboard sensors/gauges may comprise a single sensor/gauge or a plurality of sensors/gauges and sensor/gauge types, including but not limited to positional sensors such as gyroscopes, accelerometers, capacitive sensors, angular-rate sensors, shock and vibration sensors, etc., temperature sensors such as semiconductor temperature sensors, current out/voltage out temperature sensors, etc., strain gauges, force gauges, pressure gauges, flow gauges, altimeter, GPS, compass etc.

The device may be capable of recording a plurality of metrics simultaneously, including, but not limited to:

Accelerations

Velocities

Forces

Strains

Temperatures

Vibrations

Jerks

Cycles/fatigue

The onboard microprocessor communicates with the sensors and gauges and stores all of the collected raw data in the memory unit, or transfers the data to a secondary computing device (such as a smartphone, tablet, laptop, desktop computer, or equivalent, where the raw data may be processed and translated to metrics more meaningful to the user) via a wired or wireless connection (such as wired USB or serial, BlueTooth, BlueTooth Low Energy, RF radio, ZigBee radio, etc.). The device can be either directly communicating with another device(s), secondary computing device(s) or with a dedicated data receiver(s) (i.e. phone or purpose specific data receiver). The device may comprise a port or series of ports for a wired connection for data extraction and firmware/software updates.

The memory unit stores all measured and tracked kinematic data as determined by the microprocessor. The memory unit may be a flash drive, hard disk drive, solid state drive, etc. The memory unit may be permanently connected to the device housing, or removable.

The self-containing power supply of the device may be in the form of a rechargeable battery (charged either by cable such as micro-USB connector, or equivalent, or wirelessly, via inductive powering, or equivalent), or a self-generating source such as a piezoelectric supply, solar supply, kinematic generators, etc.

A single device or system of devices may be placed on various parts of the body for targeted or systematic tracking. The device may be placed in accessory holders such as bands (arm, leg, neck, etc.), belts, gloves, or may have self-adhering qualities (such as adhesives). Additionally, the device may be placed directly or with the assistance of a mounting mechanism, onto sporting equipment, i.e. bats, balls, golf clubs, hockey sticks, etc. Further, mounting equipment may be customized to the user's body or body part such that the fit of the mounting equipment is ideal—such as using a 3D scan of the body part/body of interest to create a custom molded mount. The user may communicate where the device is placed, to the device, or the system may recommend placement based on the desired exercise, movement, activity, etc. Placement of the devices may be optimized based on the desired activity to be tracked. For instance, tracking of the:

Lower body may optimally comprise devices placed in some or all of the following locations: torso, thigh, knee, ankle, and forefoot.

Upper body may optimally comprise devices placed in some or all of the following locations: torso, shoulder, elbow, and forearm.

The user interface of the external computing unit may allow the user to specifically communicate where the device is placed on the body. Alternately, the device may contain algorithms to self-determine where the device was placed.

The user interface of the external computing unit may allow the user to interact with the processed data, such as to view representations of motion from different angles and speeds, to compare motions against a baseline, to compare motions to those of other users, etc. In all of these interactions, comparative metrics may be extracted from the data, such as for comparing speeds, accelerations, forms/postures, etc. across various measurements either from the same of between users. These interactions may be in the form of visual models, numerical/graphical models, or equivalent. Captured motions and motion standards may be modified by the user (or a trainer, etc.) by a number of methods including, but not limited to: direct scaling of a motions coordinates and/or time points, modification of key extracted motion elements of subsets of a motion (i.e. angles, distances, speeds, accelerations), extension of a motion beyond its captured range via extrapolation (or equivalent) or by manual modification (i.e. wireframe node or element drag and drop for at least one time frame in a motion profile), capturing motion directly (i.e. by a trainer, professional athlete, or equivalent) with software or manual manipulation to scale and translate such a motion for fit with the intended user. One skilled in the art can contemplate other ways to substantively modify motions such that the input motion standard is not materially equivalent to the output modified motion standard. (Any such manipulation, other than basic smoothing to “clean-up” a captured motion is referred to as a modified motion standard in the context of this invention.) Motion standards which deviate from the captured motion of the intended user may be manually or algorithmically broken down into a subset of motion standards which gradually transition a user's motion to that of the final intended motion standard. A database may store historical raw data and associated metrics for future comparisons, and data may be optionally made public by the user to create a knowledgebase for creating stratified data sets, which users can access to compare metrics against. Rather than a one-size-fits-all scheme, or comparison to one's own previous motions or those of another individual or type, the ability to specifically and uniquely modify a captured motion allows for users, patients, etc., to work towards a motion standard which is catered directly to their specific needs. Certain users, patients, etc., may be at different stages along their progress toward achieving a specific motion profile, or may have limitations that prevent them from achieving a specific motion (such as injured rehabilitation patients). Custom motion standards allow for feedback to be used to adjust the approach taken towards reaching a specific motion. Additionally, many motions may be unique to a specific patient or specific activity, therefore the ability to create, modify, and store a dynamic library of modified and unmodified motion standards is critical to creating an ecosystem whereby any user's motion may be trained towards a new motion, without the dependence on a static standards. Finally, comparison to externally sourced motion standards, such as in the case of one user to another, appears to be a useful function that has often been contemplated, however, without the ability to substantively modify a motion profile to account for body, kinematic element, size and motion speed differences the utility of this feature is limited given the greatly diverse nature of the human physiology. What has not been reduced to practice is a system that has the features of contemplated by this invention, nor is it obvious why such features are critical until one tries to compare motion profiles between different users.

The baseline may be determined in a plurality of ways, including, but not limited to:

Using an individual's own historical data

A specified custom baseline as determined by the individual, coach, trainer, etc. which may or may not be based on any historical data saved for a specific user or derived from an algorithmic motion profile matching feature against motion standards stored in an external database

A user triggered event such as a voice command, a screen tap, a motion (a flick, punch, twist, etc. by the body part to which the device is attached)

Automatically determined through the software, algorithmically

As described above, certain movements of a famous athlete may be recorded using an embodiment of the disclosed device. These movements may be further published and distributed to fans of the athlete who can compare their movements to the movements of the athlete. In addition, virtual reality overlays may be used with heads up displays, screen outputs, and the like. For example, video from the game or single moment in the game may be replayed by fans and the fans may put themselves within the position of the athlete and attempt to mimic their movements. In such a case, the user may choose to use an algorithmically modified motion profile that allow effective comparison between the current motion and another modified motion profile which accounts for the user's size, speed and physiological differences.

Further, for improved accuracy and translation between different applications of a single motion standard (e.g. how a motion standard of tall user is interpreted in the analysis and comparison to the movement of a shorter user), the user may input custom metrics into the software such as height, weight, dimensions of relevant body parts, etc. Alternately, many of these metrics could be derived form camera based vision analytics of a specific user. Such metrics may allow for more translatable, and comprehensible, comparisons between users and more accurate analysis by the algorithms developed for processing and presenting the data. For example, a comparison mode may present a similarity metric between two user's golf swings, however, the current user may have a rotator cuff injury that limits the height to which they can raise their arm. If compared directly, the system may correctly present that there is a very poor match between the users strokes. However, if the user modifies the motion profile to which the comparison is being made to exclude the first and last 10% of the stroke, they may now achieve a much higher level match to the profile, allowing for specific focus on motions that are deemed important or appropriate for a specific user. This is also particularly important in the case of injury rehabilitation in physical therapy. For example, one may have a neck injury where rotating ones head from side to side is the prescribed motion. If one uses a generic, external motion profile, the physiologic differences between the bodies make comparison hard, if not useless, or alternately a recorded motion profile of their own may not have sufficient range of motion to properly instruct and gage the user's rehabilitation progress over time. In this case, the ability to modify either of these motion profiles to either compensate for physiologic difference or to extend the currently recorded range of motion for the patient provides a new modified motion profile to which useful comparison and progress metrics can be drawn.

Specific applications of the invention are listed below:

Running: measuring pronation, supination, foot strike, heel striking, impact forces, fatigue (cycle counting), etc.

Soccer: kicking and running moves/motions,

Golf: mechanics of swing for various clubs and on various slopes of ground, etc.

Tennis: mechanics of swing, foot placement

Baseball: mechanics of a pitcher's throw, mechanics of a batters swing, accelerations felt during a collision or strike to the head, etc.

Basketball: mechanics of shot, repeatability of free throw form,

Football: mechanics of a quarterback's throw, false start measuring device for linemen, snap time and form for snapper, angle of toss for snapper, accelerations felt during a collision or strike to the head, acceleration and acceleration changes of wide receivers, changes in peak speeds by wide receivers or running backs, etc.

Hockey: mechanics of puck strike

Boxing: speed and form of punches, tracking of foot placement,

Horse Racing: posture, weight distribution, etc.

Bicycling: rate of energy expenditure, posture, etc.

Swimming: stroke mechanics, etc.

Rehabilitation: any prescribed exercise, etc.

Detail of use for running application included in operation of first embodiment below.

Additional specific features of the invention are listed below:

Tracking locations of individuals or groups of individuals (i.e. members of an athletic team) for analyzing group formations, accuracy of and deviations during play executions, fatigue of players

Monitoring total energy expenditure and expenditure periods for preventing an individual from burnout and providing them with a management tool for spending energy

Remote coaching and feedback

Feedback by the device may be provided in any of the following forms:

Visual data on the screen of the device or on screen of a secondary device (i.e. smartphone)

Wire frame mannequin replays motion

Scale 0 to 100 or red to green

Numerically with safe, baseline, or other range reported

Audio feedback from the device or secondary device (i.e. smartphone), from speakers or from headphones:
Intensity of sound
Type of sound
Duration of sound

Data may be replayed for various purposes:

Track a winning shot or swing and replay it for user (virtual avatar)

Replay famous athlete's winning shot or entire game

Share with friends/family

Share data with a company

Data aggregation in the cloud or on a database may be used for a plurality of purposes, including but not limited to:

Determining common injuring

Determining ideal form, motions, etc. for optimizing efficiency, safety, peak performance, etc.

Determining common mistakes

Mimicking of an externally defined motion

In FIG. 1A the device is shown placed in a band. In such an embodiment the device may be placed on the arm, leg, or equivalent of the user. The device is activated by motion or by a switch, button, or equivalent, either on the actual device, or on a secondary computing device. The onboard sensors may then begin to record motion and other data (i.e. temperature, strain, etc.) and can either store the data on the onboard memory unit or can relay the data, to the secondary computing device, either one on the person of the user, or

Such devices may be placed in a custom insole, or equivalent. Both devices in this instance would have some combination of accelerometers, gyroscopes, and strain gauges, such that the angle of liftoff, landing, pronation, supination, foot rotation, torque, force exerted during liftoff, landing, etc. could be determined. Such a system of devices may be used on both feet for a more systematic approach. The data from the device may be sent real time to the individual for immediate feedback, or stored until after the run for a post exercise analysis. The above mentioned metrics would be reported to the individual, with the option of comparing to a specific baseline, including but not limited to, a past running session by the same individual, a past or concurrent running session by another individual, a past or concurrent running session by a group of individuals, etc.

All of these metrics would be uploaded to a cloud database for future reference, and would be used for creating a resource for other individuals performing similar exercises to interact with and learn from. The online database would not only have sharing between user capability for comparison purposes but also allow the motion of experts in any field be downloaded and used as a baseline and then be used in an unmodified or modified form. Thus, one could be able to download a sport star's motion profile (such as Tiger Wood's or LeBron James) and then compare your motion to his in real time, immediately following a movement or much later, after the event.

Embodiments of the invention are further illustrated in the foregoing examples.

### EXAMPLES

### Example 1

### Running

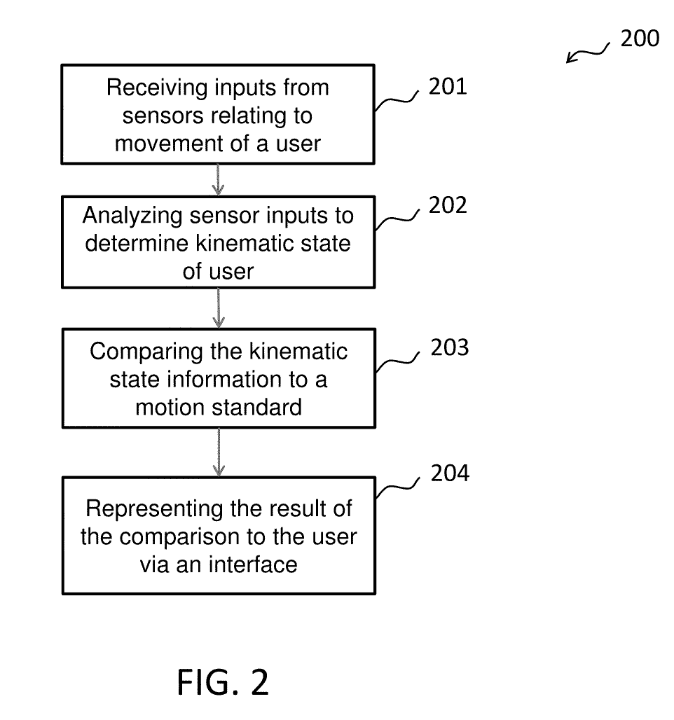

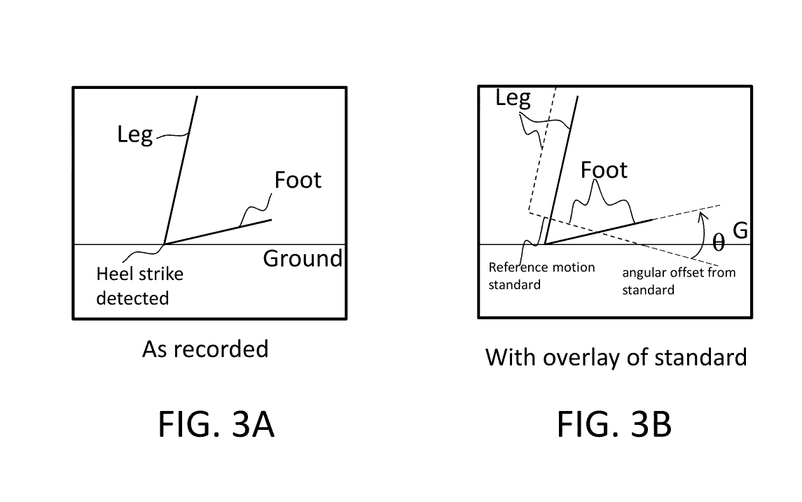

In the case of running one embodiment would comprise a series of devices placed on the foot such as at the front of the foot and at the heel of the foot and secondary computing device(s) which analyzes and algorithmically analyzes a motion compared to a motion standard as shown in the embodiments of FIG. 1B. An example of movement compared between a user's movement and that of a reference motion standard is shown in FIG. 3A and 3B. FIG. 3A shows the user's leg and foot positions when the heel strikes the ground, as detected by the sensors. Comparison of this data with a reference motion standard at a specific point along the motion profile is shown in FIG. 3B, where the offset angle θ of the foot with reference to the motion standard is shown. Software in the secondary computing device may then algorithmically modify the user's original motion to create a new motion standard unique to the user which combines information from the user's original motion and that of the reference motion standard. Such a new motion standard may take into account subtleties of the users original captured motion such as to not encourage motions which may be detrimental (i.e. overextension), and may take into account any additional factors such as user's body metrics (height, weight, age, etc.).

### Example 2

### Physical Therapy

In the case of physical therapy for rehabilitation of walking one embodiment would comprise a series of devices worn on both legs of a patient, secondary computing device(s) which analyzes a motion standard, and a physical therapist who manually adjusts the captured motion to create a unique new motion standard. Sensors would track and capture the motion of the patient while walking patient in a pre-rehabilitated state. The pre-rehabilitated motion of the patient may then be analyzed and converted to a graphical wireframe model of the captured motion by a secondary computing device to create a pre-rehabilitated motion standard. A physical therapist or equivalent may then directly manipulate the patient's wireframe pre-rehabilitated motion standard to create a new motion standard which may be overlaid with the original pre-rehabilitated motion standard or overlaid in real-time with additional captured motion of the patient. The physical therapist may create multiple unique new motion standards which increment towards a desired motion allowing the patient to gradually work towards reaching such an ideal motion and may compensate for unforeseen changes in the patients motion throughout the rehabilitation process.

While the invention has been disclosed with reference to certain embodiments, it will be understood by those skilled in the art that various changes may be made and equivalents may be substituted without departing from the scope of the invention. In addition, many modifications may be made to adapt to a particular situation or material the teachings of the invention without departing from its scope.

What is claimed is:

1. A system for measuring, tracking, and capturing a user's body kinematics comprising:
at least one sensor configured to transduce and provide kinematic information about a movement of the user's body; and
a computer interface device with processor, memory and display configured to analyze the kinematic information about said motion, wherein,
the computer interface device is configured to provide a means of modifying a motion standard, and compare the kinematic information to said modified motion standard; and
output the results of the comparison through the display

2. The system of claim 1, wherein the sensor is a gyroscopic sensor, an accelerometer, a strain sensor, or a pressure sensor.

3. The system of claim 1, wherein the motion standard is determined by recording the user's own movement.

4. The system of claim 1, wherein the motion standard is determined by manual or automatic programmatic modification or manipulation of a recorded movement.

5. The system of claim 1, where the motion standard is determined by artificially defining a motion

6. The system of claim 1, wherein the motion standard is downloaded from a database of other users' motion standards.

7. The system of claim 1, wherein the motion standard is peer-to-peer shared between users.

8. The system of claim 1, wherein the analyzing kinematic information compensates for body differences between that of the motion standard and that of the user.

9. The system of claim 1, wherein the computer interface device comprises a mobile phone, a laptop computer, or a wearable computer.

10. The system of claim 1, wherein the sensors communicate to the computer interface device via wired or wireless communication.

11. The system of claim 10, wherein the wireless communication uses a protocol selected from one of radio frequency, Bluetooth, Zigbee, Ultra Wide Band or WiFi.

12. The system of claim 1, wherein the means of modifying a motion standard is a person dragging a wire frame node in a specific time point from one location to another and then the computer algorithmically modifying the preceding and following time points such that the newly generated modified motion profile passes smoothly through the new node location generated from the user drag.

13. A method of measuring, tracking, and capturing a user's body kinematics comprising:
receiving inputs from sensors relating to movement of a user;
analyzing the sensor inputs to determine kinematic state information of the user;
comparing the kinematic information to that of a motion standard; and
representing the result of the comparison to the user via an interface.

14. The method of claim 13, wherein the motion standard comprises a database of the user's own movement.

15. The method of claim 13, wherein the motion standard comprises a database of movement of individuals other than the user.

16. The method of claim 13, wherein the motion standard comprises an artificially created motion standard.
